(19) United States
(12) Patent Application Publication
(10) Pub. No.: US 2022/0257928 A1
(43) Pub. Date: Aug. 18, 2022

(54) SYSTEM AND METHOD OF VISUAL-CORTICAL PROSTHETICS

(71) Applicant: Avtonomnaia nekommercheskaia organizatsiia «Nauchno-proizvodstvennaia laboratoriia «Sensornye tekhno, Moskva (RU)

(72) Inventors: Denis Sergeevich KULESHOV, Moskva (RU); Aleksandr Victorovich POPOV, Moskva (RU); Andrei Mikhailovich DEMCHINSKII, Stavropolskiy kray (RU); Nikolai Nikolaevich KIRICHENKO, Moskva (RU); Evgenii Konstantinovich POPOV, Moskovskaya obl (RU); Andrei Pavlovich BYTEISHCHIKOV, Moskva (RU); Mark Victorovich ZOLOTAREV, Moskva (RU)

(21) Appl. No.: 17/564,376

(22) Filed: Dec. 29, 2021

(30) Foreign Application Priority Data
Feb. 15, 2021 (RU) 2021103724

(51) Int. Cl. A61N 1/05; A61N 1/378; A61N 1/372

(52) U.S. Cl. CPC A61N 1/0531; A61N 1/37247; A61N 1/37229; A61N 1/3787

(57) ABSTRACT

The invention refers to bioengineering technologies and can be used in medical practice to restore visual functions in people who have completely lost them and allows to reduce the user's adaptation time to a new visual experience, expanding the system functional capabilities, as well as increasing the safety of use. The system contains an external part and an internal part. The external part effectively processes the video signal and sends commands to the electrodes implanted in the user.

### FIELD OF TECHNOLOGY

The proposed group of inventions refers to bioengineering technologies and can be used in medical practice to restore visual functions in people who have completely lost them.

### LEVEL OF TECHNOLOGY

Solutions aimed at restoring visual function of patients by brain neurons stimulation are known from the level of technology.

This possibility is due to the fact that the neurons that send visual signals from human eye are directly connected to the visual area V1 of the cerebral cortex. V1 cortex area consists of neurons that are responsible for contrast, color, depth, orientation, movement of the visual stimulus. Direct stimulation of specified neurons by weak electrical impulses causes the appearance of phosphenes arising in the visual field, which enables so-called phosphenic vision, capable to some extent of replacing ordinary vision for a person who has lost this ability.

Prostheses, which are permanently implanted in the human body and stimulate the visual cortex cells, are used for stimulation.

For example, a cortical visual prosthesis is known (US 2014222103 A1, Aug. 7, 2014), which is an outer and inner part placed on a flexible substrate, the inner implantable part contains stimulating electrodes, and the outer part contains a coil designed to receive and give a signal to the electrodes.

The device and method of visual prosthetics are also known (U.S. Pat. No. 5,159,927 A, 3 Nov. 1992). In this solution, the signal from video camera is processed, converted and transmitted via wire channel to the optic nerve. This solution has an increased complexity of implementation and is very inconvenient for user in operation.

The solution known from (U.S. Ser. No. 10/052,478 B2, 21 Aug. 2018) can be taken as an analog of the invention. In this solution, the signal from external video camera is converted by processor and transmitted to receiving coil of the cortical implant via another transmitting coil, with the coils located in projection of each other in immediate vicinity.

This solution has low useful functionality, because it does not allow, in particular, to carry out the object recognition, determine the distance to recognized objects and provide a user with information on surrounding objects, which may, however, be necessary in the case of low-resolution visual implants.

Thus, a common disadvantage of the technology level is the low functionality and efficiency of reproduction of environmental objects, leading to a long adaptation time of user to the possibility of ineffective recognition of visual images of phosphenic vision.

The proposed group of inventions is aimed at overcoming the disadvantages of the technology level and achieving technical results consisting in reducing the user's adaptation time to the new visual experience, in expanding the functional capabilities of the system, as well as increasing the safety of use.

### SUMMARY

To achieve the specified technical results, a group of inventions is proposed, which in a generalized form includes a system and method of visual-cortical prosthetics.

According to the first aspect of the invention, a visual-cortical prosthesis system consisting of an external part and an implantable part is proposed, where the implantable part is an interconnected receiving antenna in a biocompatible silicone housing, an electrode control chip enclosed in a titanium housing, and a matrix of electrodes made of conductive material on biocompatible substrate. And the outer part consists of the first device designed to be placed on the user's head with an adjustable hoop, and the second device is a video signal processing unit, and the hoop is equipped with two video cameras built into its front part, a power supply, a transmitting antenna, a microcontroller, a memory and an interface for connecting a processing unit. And the video signal processing unit is a microcomputer located in the housing, made with the ability to further process the video signal to identify objects and issue signals based on the results of processing, as well as: Wi-Fi, Bluetooth, power modules, interfaces for charging, connecting external audio devices, connecting to the hoop, and also control elements.

According to the second invention aspect, a method for operating the system of visual cortical prosthesis is proposed, which includes receiving a video signal from video cameras in the first device, its linear recoding into commands for stimulating electrodes and generating a signal of electrode stimulation, and upon receipt of a corresponding command, activating additional processing of the video signal in the second device associated with the first device which includes: recording a video frame stream, conversion of video frames into a pattern of averaged signals with a resolution corresponding to the size of electrode matrix, detection of target object boundaries, recognition of objects, creation of a depth map, determination of distance to objects, based on processing results formation and output of the stimulation signal of electrodes with simultaneous sounding of information obtained as a result of object recognition and/or determination of distance to objects.

The following is a more detailed description of the proposed group of inventions in its preferred embodiments.

### Implementation of the Invention

A cortical visual prosthesis (CVP) is a system consisting of a set of external and internal (implantable) devices designed to artificially activate useful visual sensations in totally blind people, i.e. to prosthetize vision.

### BRIEF DESCRIPTION OF THE DRAWINGS

For a more complete understanding, reference is made below to the corresponding explanatory drawings which show the components of the proposed system.

According to the drawings, the proposed system contains:

FIG. 1 shows the head device in the form of a hoop;

FIG. 2 shows the video signal processing unit (additional processing);

FIG. 3 shows the block diagram of video signal processing unit elements;

FIG. 4 shows an implanted part, hereinafter referred to as implant or prosthesis.

FIG. 5 shows a scheme of the implant use.

### DETAILED DESCRIPTION OF THE PREFERRED EMBODIMENT

The visual cortical prosthesis system consists of external and implantable parts.

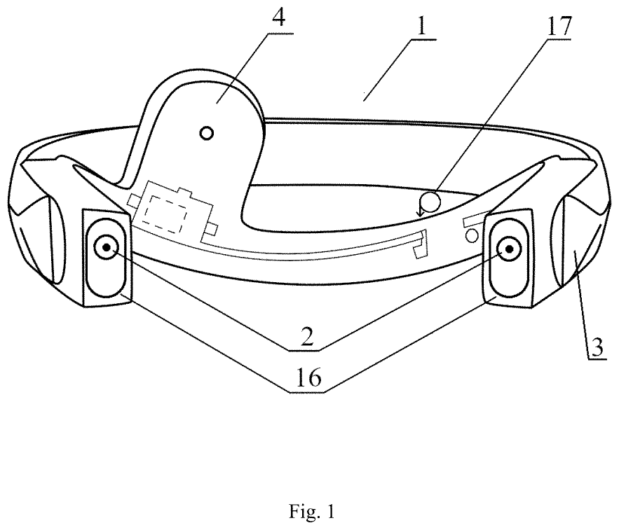

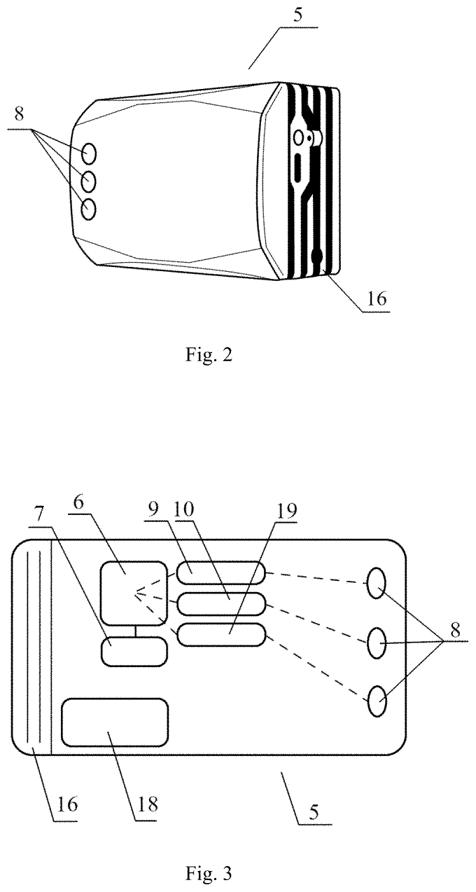

The external part includes a first device designed to be placed on the user's head being an adjustable hoop (FIG. 1), and a second device being a video signal processing unit (FIGS. 2,3).

As shown in FIG. 1, the head hoop or head device includes:
instrument for converting images into a video signal, made in the form of two video cameras 2 built into the front of the hoop 1. Video cameras 2 are video recording devices, and are necessary to obtain images and a map of the surrounding space depth, their location in the front of the hoop is determined by their purpose.
built-in microcontroller 3, which is part of the device hardware, designed to convert the video signal into linearly recoded commands to stimulate the electrodes, and these commands are transmitted by antenna 4, and also designed to transmit the video signal through a cable (not conventionally shown in the figure) to the video signal processing unit 5, where it is converted into personalized stimulation patterns, which, after returning to the head hoop 1, are redirected to the cortical implant 11 using the above antenna 4,
antenna 4, which is an external transmitting radio coil required for transmission of signals and power,
cooling radiators 16, located in the frontal part of the hoop 1, made in the priority embodiment of aluminum and designed for cooling;
power supply (not conventionally shown in Fig.) in the form of two batteries, serving for autonomous operation of the device for a period of time determined by the battery capacity;
communication interface 17—USB-C port of connection to the video processing unit 5 and charging device, located on the back side of the hoop 1 (in FIG. 1, the location shown conventionally).

FIG. 2, FIG. 3 shows the second external part device—the video signal processing unit 5 of the cortical visual prosthesis 11, in which the signal converted by microcontroller is further converted into an intelligent version of the stimulus set, and other intelligent signal processing is performed. The specified unit includes:
processor 6, which is part of the device hardware, provides execution of commands and functionality of related blocks (stimulation parameters control and mode switching block 9, smart search mode activation block 10, power-on block with charge indication 19) at the software level,
RAM block 7, which is part of the device hardware, necessary for temporary storage of information received and transmitted within the cortical visual prosthesis, closely related to the processor 6 when implementing the operation of software level components,
control buttons 8 designed to activate the stimulation parameter control and mode switching unit 9, the smart search mode activation unit 10, and the power-on unit with charge indication 19,
a block for controlling stimulation parameters and switching modes 9, made by semantic section of the software level based on the processor 6, designed for changing the modes of object contouring and selecting parameters of electrode stimulation. To perform this functionality, two rules are used: one—for selecting silhouettes and masks of significant objects, and the other—for selecting the basic structural lines that delineate the room boundaries. The implementation of this functionality does not require an Internet connection, it is performed autonomously,
smart search mode activation unit 10, made by the semantic section of the software level based on the processor 6, designed to find the object position in the information data received from cameras, and its classification. When implementing the functionality, it uses a machine vision algorithm based on a pre-trained neural network algorithm, for better perception of objects and space in various situations by the prosthesis and the user, respectively. Additionally, unit 10 is equipped with the function of sounding out the found object to the user via any compatible headset for accelerated adaptation of the brain to images received by electrode signal,
power unit with charge indication 19, made by semantic section of the software level based on the processor 6, designed to turn on and off the device and to notify the user about the batteries 18 charge level,
radiator 16, made in the priority embodiment of aluminum, designed to cool the hardware,
battery 18, designed for autonomous operation of the device for a period of time determined by the battery capacity.

Thus, the video signal processing unit is a microcomputer placed in the housing, made with the ability to further process the video signal to identify objects and issue signals based on the results of processing, as well as: modules Wi-Fi, Bluetooth, power, interfaces for charging, connecting external audio devices, connecting to the hoop and control elements.

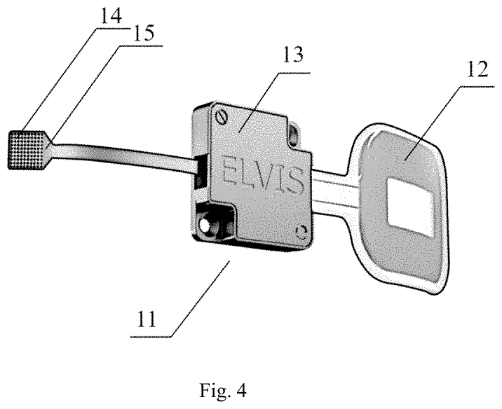

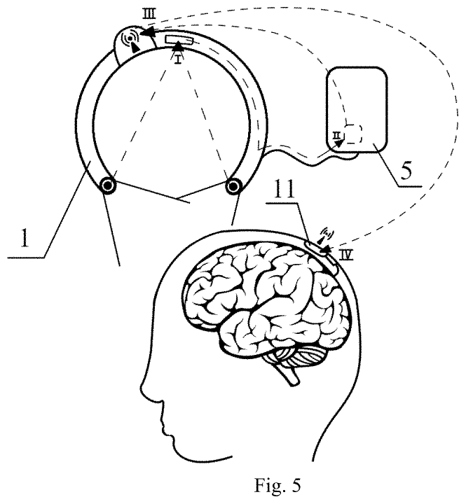

Implantable part is a cortical implant 11 of the cortical visual prosthesis, permanently implanted in the human body and intended for direct stimulation of cells of the visual cortex of the brain, is shown in FIG. 4.

The cortical implant 11 includes:
antenna 12, which is the receiving radio coil necessary to receive power and functional stimulation commands for the electrodes in the form of signals,
electrode control chip 13, which is a pre-stitched component prior to implantation, a receiver board for receiving data and commands, located in the housing, predominantly made of titanium.
electrode matrix 14, immersed in an inert flexible base 15, directly executing the received commands, acting with small currents on the visual cortex neurons of the brain.

Thus, the implanted part consists of a receiver antenna 12 in biocompatible silicone housing, an electrode control chip 13 enclosed in titanium housing, and a matrix of electrodes 14 made of conductive polymer and immersed in an inert flexible base 15.

As shown above, the system has two independent structural units—a removable external one, consisting of a hoop with two cameras and a transmitting radio frequency coil, as well as a plug-in video signal processing unit and for image recognition based on artificial intelligence algorithms; and a chronic internal one, consisting of an electrode array with electrode ends, a chip for signal conversion and a receiving antenna.

The external part of the system is applied to receive visual information on the environment, its processing and transmission to the implanted part. The external part elements are preferably connected via the USB/USB-C interface.

Two cameras 2 are built into the hoop to capture images and a depth map of the surrounding space. It also has a mounted antenna 4, which transmits power and functional stimulation commands to the implant 11 for the electrodes in the form of personalized stimulation patterns. In the software plan, the hoop 1 has pre-stored algorithms to process the signal from camera and transmit it to the electrodes without additional intelligent processing with a help of the video signal processing unit 5. This processing can be carried out by the microcontroller 3 contained in the hoop. For more comfortable use, the hoop 1 can be equipped with cooling radiators 16 placed in the front, where video cameras 2 are mounted, as shown in FIG. 1.

In the video signal processing unit 5, the signal from the video cameras 2 is converted into a set of stimuli understandable to the prosthesis, and additional signal processing and issuance is performed, as shown below. The unit has functional control buttons: power, intelligent object search and video signal processing mode for the prosthesis for a better perception of space in different situations.

The inner part (cortical implant) 11 is permanently implanted into the human body and is designed to directly stimulate the visual cortex cells. This part includes: an antenna 12 that receives power and functional stimulation commands for the electrodes; a chip 13 to control signals from the external part of the system; and a surface implant, which is the electrode array 14 immersed in the inert flexible base 15.

The system functions as follows.

Functioning of the system is possible only when the hoop 1 is placed on the user's head, when the external transmitting and internal receiving coils are in projection of each other and in close proximity enough to transmit the control signal and power.

In a preferred embodiment, the implant is an interconnected receiving antenna in a biocompatible silicone housing, an electrode control chip enclosed in a titanium housing, and a 10×10 (100 electrodes) electrode matrix of conductive material on a biocompatible substrate.

All implant elements are made of safe materials for the body, which ensures the possibility of its chronic use.

The antenna 12 and chip 13 are mounted on the skull under the scalp, while the electrode array 14 is immersed in the cranial cavity and placed on the brain surface in the visual cortex projection—areas V1-V2-V3 on the medial surface of one brain lobe, responsible mainly for activation of the central visual field. The cortical implant has separate software that performs the functions of receiving and interpreting the digital signal received through the coil, as well as controlling the analog supply system and controlling the power to the electrode array.

Generally, the image from one of the hoop cameras is linearly encoded by microcontroller and converted into electrode stimulation signals (commands) transmitted by the transmitting antenna, which causes phosphenes that appear in the user's field of view. This function may be sufficient in case of identification of large objects. However, if the user needs to identify medium and small objects, it is necessary to use additional intelligent features.

As a result, the invention proposes an additional direction of the video signal to the video signal processing unit.

In the video signal processing unit 5, the image is averaged to 100 pixels into a stimulation pattern according to one of the selected patterns. Here the image is also analyzed in real time using artificial vision algorithms to broadcast a sound description of the observed scene (its individual objects) to the user. After processing the video signal, the stimulation pattern is sent directly back to the implant via its own radio transmitter to transmit information via electromagnetic radiation in the radio frequency range to a receiving antenna connected to a chip powered from the hoop by wireless energy transfer via a special coil and fixed with screws under the scalp to the cranial box. From the receiving coil, the signal is sent to the chip, where it is converted into small currents that have the properties necessary for safe and effective stimulation of the visual cortex neurons. The final device element is the electrode array having 100 electrode endings immersed in dielectric material and located on the medial surface of the visual cortex in the projection of the calcarine sulcus V1.

For example, the signal from video cameras is converted into a set of stimuli understandable to the prosthesis: a stream of color video frames at a frequency of 30 Hz and 640*480 pixels is recorded from the camera, and then converted into a pattern of averaged signals with a resolution of 10*10, which corresponds to the electrode matrix.

In the software plan, the unit uses machine vision algorithms based on artificial intelligence algorithms to identify specific objects (people, road signs, household items, etc.). When processing the image, 2 algorithms are used in parallel, one—for the selection of silhouettes and masks of essential objects and the second—for the selection of basic structural lines defining the room boundaries.

The proposed system uses the principle of the most economical exposure by the number of electrodes and the duration of their activation with the use of preliminary computer image processing with the help of a microcomputer. This approach optimizes the visual and geometric properties of the camera image and performs semantic analysis of the information presented in the picture in order to recognize and select visual objects of interest. Furthermore, an additional feature is the function of object recognition using a pre-trained neural network algorithm with the function of sounding out the found object for accelerated brain adaptation to the images obtained with the help of electrode signals. Neural network training can be performed using the neural network deep learning library. Object recognition can be performed using one of the available neural network architectures, which allows to classify and find the position of an object in the image. Relevant databases can be preloaded into the microcomputer memory, so the system can operate offline without an Internet connection.

The method of the proposed system operation is a set of sequential methods of video signal processing from video cameras for visual prosthetic systems, and is designed to improve the experience of users of visual prosthetic systems in the form of contouring of target objects, their identification using machine vision and artificial intelligence algorithms, as well as determining the distances to physical objects of the observed scene with the provision of feedback in the form of sounding and/or vibration response.

The method includes both the linear coding mode noted above and an activated additional processing mode. This mode can be activated via the intelligent object search mode activation button located on the video processing unit, which generates the corresponding control command.

The method steps in a particular implementation include the following processing stages.

A stream of color video frames is recorded with a frequency of 30 Hz and the size corresponding to the resolution of the used camera, for example, 640*480 pixels. This image undergoes a series of transformations to prepare the definition of boundaries, after which the algorithm for detecting the boundaries of target objects is applied. Then a number of transformations are performed with each obtained frame with the object boundaries, as a result of which the image is averaged to the number of electrodes in the used electrode, each of which corresponds to the necessary currents and time parameters of influences. The step does not require an Internet connection and can be used offline.

With each frame, a series of transformations and calculations are performed using trained computer vision algorithms (or other artificial intelligence algorithms), which result in alerting (by sounding) the user of the prosthetic visual system to the presence of certain objects in the observed scene. The stage does not require an Internet connection, as long as there are trained databases in memory.

Direct processing of video signals from cameras, highlighting contours, object recognition and determination of distances to physical objects. For this method, one or two video cameras should be present in the visual prosthesis. The method synchronously analyzes data from two or more cameras to create a depth map and calculate distances to physical objects. The user can be alerted of the distance to the object. The step does not require an Internet connection and can be used offline. The user can be simultaneously notified both about the presence of certain objects and the distance to them.

Wi-Fi or Bluetooth modules built into the control unit can be used to update databases and/or perform process steps that require a network connection.

Thus, the description shows how a group of inventions can be implemented using known means from the technology level.

The group of inventions makes it possible to significantly reduce the user's adaptation time to the new visual experience by implementing a new function—additional processing of the video signal and parallel voicing of information related to the recognized objects of the environment. This also allows the system to be utilized by users of low-resolution prosthetic vision systems. The safety of use is ensured by the materials applied to implement the devices. In addition, the safety of use follows from the ability to voice information to the user about the distance to the objects.

Manufacturing and testing prototypes of the system products showed their high efficiency and the possibility to use them for medical purposes for their intended purpose.

What is claimed is:

1. A visual cortical prosthesis system, comprising: an external and an implantable parts:
the implanted part comprising a receiver antenna in a biocompatible silicone housing, an electrode control chip enclosed in a titanium housing, and a matrix of electrodes made of conductive polymer and immersed in an inert flexible base, all of them connected with each other,
and the external part consists of a first device designed to place an adjustable hoop on a user's head, and a second device being a video signal processing unit,
moreover, the hoop is equipped with two video cameras, a power supply unit, a transmitting antenna, a microcontroller, a memory, and an interface for connecting a processing unit built into a front of the hoop, and
the video signal processing unit is a microcomputer placed in a housing, the video signal processing unit processes a video signal to identify objects and issue signals based on results of the processing, as well as: Wi-Fi, Bluetooth, power modules, interfaces for charging, connecting external audio devices, connecting to the hoop, and control elements.

2. A method for operating the system according to claim 1, which includes receiving a video signal from video cameras in the first device, its linear recoding into commands for stimulating electrodes and generating a signal of electrode stimulation, and upon receipt of a corresponding command, activating additional processing of the video signal in the second device associated with the first device which includes: recording a video frame stream, conversion of video frames into a pattern of averaged signals with a resolution corresponding to a size of electrode matrix, detection of target object boundaries, recognition of objects, creation of a depth map, determination of a distance to objects, based on processing results formation and output of the stimulation signal of electrodes with simultaneous sounding of an information obtained as a result of object recognition or determination of distance to objects.
